(19) United States
(12) Patent Application Publication
(10) Pub. No.: US 2006/0182796 A1
(43) Pub. Date: Aug. 17, 2006

(54) Taste masked pharmaceutical compositions

(75) Inventors: Chuanbin Wu, Weston, FL (US); Harold Injety, Coral Springs, FL (US); Tim Weng, Cooper City, FL (US)

Correspondence Address: PATREA L. PABST;PABST PATENT GROUP LLP, 400 COLONY SQUARE, SUITE 1200, ATLANTA, GA, 30361, US

(73) Assignee: ABRIKA PHARMACEUTICALS, INC.

(21) Appl. No.: 11/346,700

(22) Filed: Feb. 3, 2006

Related U.S. Application Data
(60) Provisional application No. 60/649,644, filed on Feb. 3, 2005.

(51) Int. Cl. A61K 9/48; A61K 9/20

(52) U.S. Cl. 424/451; 424/464

(57) ABSTRACT

A pharmaceutical composition for oral administration containing a pharmaceutically active ingredient coated with an amount of a polymer combination of an enteric polymer and an ammonio methacrylate copolymer to effectively mask the taste of the medicament. In a preferred embodiment, the ratio of the enteric polymer to the ammonio methacrylate copolymer is about 40:60 to about 90:10, preferably about 60:40, by weight of polymer. The pharmaceutical coating composition is soluble in the acidic environment of the stomach, which generally has a pH value of about 1.0 to 3.0, but relatively insoluble at higher pH values of the mouth. The coatings provide for rapid release and absorption of the drug after it passes through the mouth, and is particularly desirable in the case of liquid dosage forms.

### CROSS-REFERENCE TO RELATED APPLICATIONS

This application claims priority to U.S. Ser. No. 60/649,644 entitled “Taste Masked Pharmaceutical Compositions” by Chuanbin Wu, Harold Injety, and Tin Weng, which was filed on Feb. 3, 2005.

### FIELD OF THE INVENTION

This invention relates to oral pharmaceutical formulations which effectively mask the unpleasant taste of pharmaceuticals or nutritional supplements with bitter or otherwise undesirable taste characteristics.

### BACKGROUND OF THE INVENTION

Oral dosage forms are taken by patients in the form of, for example, solutions, emulsions, suspensions, capsules and tablets. Many active ingredients, such as antibiotics, possess a strong, unpleasant taste, which is why either contact of the medicinal substance with the mucosa of the mouth and pharynx is preferentially avoided or the bitter taste is masked. If the dosage form is formulated as a tablet or capsule and swallowed whole, the unpleasant taste of the medicinal substance is greatly minimized or avoided altogether since the capsule or tablet keeps the active ingredient from contacting the mouth. However, children, the elderly, and many other patients have difficulty in swallowing tablets and capsules. For such patients, pharmaceutically active ingredients are variously formulated as chewable tablets, mouth-dissolving tablets, dispersible tablets, dry powders for reconstitution, or liquid dosage forms. Even with these dosage forms, however, the possibility remains that there will be a perceptible exposure of the active drug to the taste buds; thus, a major requirement of such dosage forms is that they must be palatable. The palatability of the liquid or chewable dosage form is a critical factor in ensuring patient compliance.

In some cases, the unpleasant taste of the active medicament in a liquid or chewable formulation can be overpowered by adding flavoring ingredients and sweeteners or by coating the dosage form to improve taste and palatability. However, where the active ingredient possesses a particularly strong or bitter taste, such as is the case with many antibiotics, the mere addition of such flavoring ingredients and sweeteners is insufficient to improve taste and palatability. Accordingly, various taste masking coating compositions have been employed in the formulation of liquid suspension and chewable tablet dosage forms.

For example, WO2004052345 to Ranbaxy Laboratories, Ltd. describes coating compositions for taste masking and methods for applying the coating compositions to dosage forms to mask the taste of a medicinal substance. The taste masking coating compositions generally include a copolymer of acrylate and methacrylate with a quaternary ammonium group in combination with sodium carboxymethylcellulose and a polyvinyl alcohol-polyethylene glycol copolymer.

U.S. Pat. No. 5,489,436 to Hoy et al. describes chewable tablets made from a coated medicament where the coating is a “reverse enteric coating” designed to be soluble at the lower pH of the stomach but relatively water insoluble at the higher pH's of the mouth. The coatings are comprised of a polymer blend of dimethylaminoethyl methacrylate and neutral methacrylic acid ester and a cellulose ester.

U.S. Pat. No. 5,599,556 to Meyer et al. describes liquid formulations where the active ingredient is coated with a single outer polymeric coating derived from prolamine cereal grain proteins and a plasticizing agent. The coatings are designed to rapidly degrade once the composition leaves the mouth.

While coating methods for taste masking solid oral oral dosage forms have been developed, they have not been used in a liquid formulation, where the taste masking coating will need to survive in an aqueous environment for an extended period of time.

It is therefore an object of the present invention to provide a taste masking formulation suitable for use in both a solid dosage form and an aqueous liquid suspension wherein the formulation is stable and retains its taste masking properties in an aqueous environment over an extended period, yet which exhibits immediate bioavailability after swallowing and ingestion.

### BRIEF SUMMARY OF THE INVENTION

Pharmaceutical compositions for oral administration are provided that contain a pharmaceutically active medicament coated with a combination of an enteric polymer and an ammonio methacrylate copolymer effective to mask the taste of the medicament in the mouth, but which is rapidly soluble under the conditions found in the stomach. In the preferred embodiment, the polymer weight ratio of the enteric polymer to the ammonio methacrylate copolymer is between about 40:60 to about 90:10, preferably about 60:40. The pharmaceutical coating composition is soluble in the acidic pH of the stomach, which is generally about 1.0 to 3.0, but relatively insoluble at the non-acidic pH of the mouth, which is typically about 5.0. The coatings provide for rapid release and absorption of the drug, which is generally desirable in the case of liquid dosage forms.

### BRIEF DESCRIPTION OF THE DRAWINGS

FIG. 1 is a graph of the dissolution (% L.C. Dissolved) of doxycycline beads prepared from Formulation C in pH 5.5 Buffer Medium and 0.1 N HCl Medium versus time (min).

FIG. 2 is a graph of the dissolution (% L.C. Dissolved) of doxycycline beads prepared from Formulation B in pH 5.5 Buffer Medium versus time (min).

FIG. 3 is a graph of the dissolution (% L.C. Dissolved) of doxycycline beads prepared from Formulation B in 0.1 N HCl Medium versus time (min).

FIGS. 4a and 4b are graphs of the dissolution (% L.C. Dissolved) of doxycycline beads prepared from Formulation A in 0.1 N HCl Medium (FIG. 4a) or pH 4.5 Buffer Solution versus time (min) (FIG. 4b).

### DETAILED DESCRIPTION OF THE INVENTION

I. Compositions

The pharmaceutical compositions contain a pharmaceutically active medicament coated with a polymer blend of an enteric polymer and an ammonio methacrylate copolymer (wherein the enteric polymer is different from the ammonio methacrylate copolymer) to effectively mask the taste of the medicament. In the preferred embodiment, the polymer weight ratio of the enteric polymer to the ammonio methacrylate copolymer is between about 40:60 to about 90:10, preferably about 60:40. The pharmaceutical coating composition is soluble in the acidic pH of the stomach, which is generally about 1.0 to 3.0, but relatively insoluble at the non-acidic pH of the mouth. The coatings provide for rapid release and absorption of the drug in the stomach, which is generally desirable in the case of liquid dosage forms. The taste masked liquid composition may be a syrup, a ready-to-use suspension, or extemporaneously prepared liquid syrup or suspension such as, for example, dry powder for reconstitution with water, liquid concentrate for dilution, dispersible tablet or capsule.

A. Taste Masking Coating

In the preferred embodiment, the taste masking polymer blend comprises about 3 to 120% by weight of the solid substrate, most preferably about 40% by weight of the solid substrate.

1. Ammonio Methacrylate Copolymers

Copolymers of acrylate and methacrylate with a quaternary ammonium group are used in the taste-masked formulations. Suitable copolymers are commercially available under the tradename Eudragit® (Rohm Pharma; Westerstadt, Germany), including copolymers of acrylates and methacrylates with quaternary ammonium groups (Eudragits® RL 100, RL PO, RL 12.5, RL 30 D, RS 100, RS PO, RS 12.5, RS 30 D). The copolymers are either highly permeable (RL 100, RL PO, RL 12.5, RL 30 D) or poorly permeable (RS 100, RS PO, RS 12.5, RS 30 D). The copolymers may be in the form of an aqueous dispersion, powder, granules, or films.

2. Enteric Polymers

Enteric polymers become soluble in the higher pH environment of the lower gastrointestinal tract or slowly erode as the dosage form passes through the gastrointestinal tract.

Preferred materials include, but are not limited to, cellulose derivatives such as cellulose in microcrystalline or powdered form, and cellulosis polymers such as cellulose acetate, cellulose succinate, and cellulose phthalate, hydroxypropylmethyl cellulose (“HPMC”) acetate, HPMC succinate, HPMC phthalate, cellulose acetate trimellitate, cellulose acetate phthalate, and combinations thereof. Additional polymers include polyvinylacetate phthalate, copoly(ethylene vinylacetate) (EVAC), maleic anhydride-co-alkylene copolymers, polyalkylene oxides, and mixtures thereof.

A second preferred group includes acrylic acid polymers and copolymers other than ammonio methacrylate polymers, preferably formed from acrylic acid, methacrylic acid, methyl acrylate, ethyl acrylate, methyl methacrylate and/or ethyl methacrylate, and other methacrylic resins that are commercially available under the tradename Eudragit™ (Rohm Pharma; Westerstadt, Germany, including anionic copolymers of methacrylic acid and methacrylate with carboxylic acid groups such as methacrylic acid copolymer, Type C (Eudragit™ L30D-55 and L100-55 (soluble at pH 5.5 and above); methacrylic acid copolymers, Type A and methacrylic acid-methyl methacrylate copolymers (1:1) (Eudragit™ L-100, soluble at pH 6.0 and above); and methacrylic acid copolymers, Type B and methacrylic acid-methyl methacrylate copolymers (1:2) (Eudragit™ S, soluble at pH 7.0 and above, as a result of a higher degree of esterification)).

In the preferred embodiment, the enteric polymer is methacrylic acid copolymer Type A, B, and C, hydroxypropyl methylcellulose phthalate, cellulose acetate phthalate, and combinations thereof.

In the preferred embodiment, the polymer weight ratio of the enteric polymer to the ammonio methacrylate copolymer is between about 40:60 to about 90:10, preferably about 60:40.

B. Active Ingredients

In preferred embodiments the active ingredients useful in the taste masked liquid formulations include β-adrenergic receptor blockers, alkaloids, antacids, analgesics, anabolic agents, anti-anginal drugs, anti-allergy agents, anti-angiogenesis agents, anti-arrhythmia agents, antiasthmatics, antibiotics, anticholesterolemics, anticonvulsants, anticoagulants, antidepressants, anti-diabetics, antidiarrheal preparations, anti-emetics, anti-epileptics, antihistamines, antihypertensives, anti-infectives, anti-inflammatory agents, antihyperlipidemic drugs, antimanics, anti-migraine agents, antinauseants, anti-parkinsonism drugs, antipsychotics, antistroke agents, antithyroid preparations, anabolic drugs, antiobesity agents, antiparasitics, antipsychotics, antipyretics, antispasmodics, antithrombotics, antitumor agents, antitussives, antiulcer agents, anti-uricemic agents, antiviral drugs, anxiolytic agents, appetite stimulants, appetite suppressants, beta-blocking agents, bronchodilators, calcium antagonists, cardiotonic drugs, cardiovascular agents, cerebral dilators, cholecystekinin antagonists, chemotherapeutic agents, cholesterol reducing agents, cognition activators, contraceptives, coronary vasodilators, cough suppressants, CNS drugs, decongestants, diuretics, drugs for erectile dysfunction, emollients, erythropoietic drugs, expectorants, fertility agents, fungicides, gastrointestinal agents, gout treating drugs, hyperglycemic agents, hypnotic drugs, hypoglycemic agents, laxatives, migraine treatments, mineral supplements, mucolytics, narcotics, neuroleptics, neuromuscular drugs, non-steroidal anti-inflammatories (NSAIDs), nutritional additives, osteoporosis treating agents, peripheral vasodilators, polypeptides, prostaglandins, psychotropics, renin inhibitors, sedatives, serotonin receptor antagonists, steroidal anti-inflammatory drugs, steroids, stimulants, sympatholytics, thyroid preparations, tranquilizers, uterine relaxants.

Antihypertensive agents include, amlodipine, benazepril, benidipine, candesartan, captopril, carvedilol, darodipine, dilitazem, diazoxide, doxazosin, enalapril, epleronone, eposartan, felodipine, fenoldopam, fosinopril, guanabenz, iloprost, irbesartan, isradipine, lercardinipine, lisinopril, losartan, minoxidil, nebivolol, nicardipine, nifedipine, nimodipine, nisoldipine, omapatrilat, phenoxybenzamine, prazosin, quinapril, reserpine, semotiadil, sitaxsentan, terazosin, telmisartan, and valsartan.

Anti-diabetic agents include acetohexamide, chlorpropamide, ciglitazone, farglitazar, glibenclamide, gliclazide, glipizide, glucagon, glyburide, glymepiride, miglitol, pioglitazone, nateglinide, pimagedine, repaglinide, rosiglitazone, tolazamide, tolbutamide, triampterine, and troglitazone.

Antiviral agents include the antiherpes agents acyclovir, famciclovir, foscamet, ganciclovir, idoxuridine, sorivudine, trifluridine, valacyclovir, and vidarabine, and otherantiviral agents such as abacavir, amantadine, amprenavir, delviridine, didanosine, efavirenz, indinavir, interferon alpha, lamivudine, nelfinavir, nevirapine, ribavirin, rimantadine, ritonavir, saquinavir, stavudine, tipranavir, valganciclovir, zalcitabine, and zidovudine; and other antiviral agents such as abacavir, indinavir, interferon alpha, nelfinavir, ribavirin, rimantadine, tipranavir, ursodeoxycholic acid, and valganciclovir.

Anti-inflammatory agents and non-opioid analgesics, such as aloxiprin, aspirin, auranofin, azapropazone, azathioprine, benorylate, butorphenol, capsaicin, celecoxib, diclofenac, diflunisal, esonarimod, etodolac, fenbufen, fenoprofen calcium, flurbiprofen, ibuprofen, indomethacin, ketoprofen, ketorolac, leflunomide, meclofenamic acid, mefenamic acid, meloxicam, nabumetone, naproxen, novantrone, oxaprozin, oxyphenbutazone, parecoxib, phenylbutazone, piclamilast, piroxicam, rofecoxib, ropivacaine, sulindac, tetrahydrocannabinol, tramadol, tromethamine, valdecoxib, and ziconotide, as well as the urinary analgesics phenazopyridine and tolterodine.

Anti-angina agents include mibefradil, refludan, nahnefene, carvedilol, cromafiban, lamifiban, fasudil, ranolazine, tedisamil, nisoldipine, and tizanidine.

Anti-arrhythmic agents include drugs such as amiodarone, disopyramide, flecainide acetate and quinidine sulfate.

Anti-asthma agents include zileuton, zafirlukast, terbutaline sulfate, montelukast, and albuterol.

Anti-bacterial agents, such as alatrofloxacin, azithromycin, baclofen, benethamine penicillin, cinoxacin, ciprofloxacin, clarithromycin, clofazimine, cloxacillin, demeclocycline, dirithromycin, doxycycline, erythromycin, ethionamide, furazolidone, grepafloxacin, imipenem, levofloxacin, lorefloxacin, moxifloxacin, nalidixic acid, nitrofurantoin, norfloxacin, ofloxacin, rifampicin, rifabutine, rifapentine, sparfloxacin, spiramycin, sulphabenzamide, sulphadoxine, sulphamerazine, sulphacetamide, sulphadiazine, sulphafurazole, sulphamethoxazole, sulphapyridine, tetracycline, trimethoprim, trovafloxacin, and vancomycin.

Antibiotics include cefuroxime axetil, cefpodoxime proxetil, ciprofloxacin, erythromycin, and clarithromycin.

Anti-cancer agents and immunosuppressants, such as alitretinoin, aminoglutethimide, amsacrine, anastrozole, azathioprine, bexarotene, bicalutamide, biricodar, bisantrene, busulfan, camptothecin, candoxatril, capecitabine, cytarabine, chlorambucil, cyclosporin, dacarbazine, decitabine, ellipticine, estramustine, etoposide, gemcitabine, irinotecan, lasofoxifene, letrozole, lomustine, melphalan, mercaptopurine, methotrexate, mitomycin, mitotane, mitoxantrone, mofetil, mycophenolate, nebivolol, nilutamide, paclitaxel, palonosetron, procarbazine, ramipril, rubitecan, sirolimus, tacrolimus, tamoxifen, teniposide, testolactone, thalidomide, tirapazamine, topotecan, toremifene citrate, vitamin A, vitamin A derivatives, and zacopride.

Anti-coagulants and other agents for preventing and treating stroke, such as cilostazol, citicoline, clopidogrel, cromafiban, dexanabinol, dicumarol, dipyridamole, nicoumalone, oprelvekin, perindopril erbumine, phenindione, ramipril, repinotan, ticlopidine, tirofiban, and heparin, including heparin salts formed with organic or inorganic bases, and low molecular weight heparin, i.e., heparin fragments generally having a weight average molecular weight in the range of about 1000 to about 10,000 D and exemplified by enoxaparin, dalteparin, danaproid, gammaparin, nadroparin, ardeparin, tinzaparin, certoparin, and reviparin.

Anti-diabetics, such as acetohexamide, chlorpropamide, farglitazar, glibenclamide, gliclazide, glipizide, glimepiride, miglitol, nateglinide, pimagedine, pioglitazone, repaglinide, rosiglitazone, tolazamide, tolbutamide, troglitazone, and voglibose;

Anti-epileptics include beclamide, carbamazepine, clonazepam, ethotoin, felbamate, fosphenyloin, lamotrigine, methoin, methsuximide, methylphenobarbitone, oxcarbazepine, paramethadione, phenacemide, phenobarbitone, phenyloin, phensuximide, primidone, sulthiame, tiagabine, topiramate, valproic acid, and vigabatrin.

Anti-fungal agents include drugs such as amphotericin, butenafine, butoconazole nitrate, clotrimazole, econazole nitrate, fluconazole, flucytosine, griseofulvin, itraconazole, ketoconazole, miconazole, natamycin, nystatin, sulconazole nitrate, oxiconazole, terbinafine, terconazole, tioconazole and undecenoic acid.

Anti-gout agents, include drugs such as allopurinol, probenecid and sulphin-pyrazone;

Antihistamines and allergy medications include acrivastine, astemizole, chlorpheniramine, cinnarizine, cetirizine, clemastine, cyclizine, cyproheptadine, desloratadine, dexchlorpheniramine, dimenhydrinate, diphenhydramine, epinastine, fexofenadine, flunarizine, loratadine, meclizine, mizolastine, oxatomide, and terfenadine.

Anti-malarials include drugs such as amodiaquine, chloroquine, chlorproguanil, halofantrine, mefloquine, proguanil, pyrimethamine and quinine sulfate.

Agents for treating headaches, including anti-migraine agents, such as almotriptan, butorphanol, dihydroergotamine, dihydroergotamine mesylate, eletriptan, ergotamine, frovatriptan, methysergide, naratriptan, pizotyline, rizatriptan, sumatriptan, tonaberstat, and zolmitriptan.

Anti-muscarinic agents include atropine, benzhexol, biperiden, ethopropazine, hyoscyamine, mepenzolate bromide, oxyphencyclimine, scopolamine, and tropicamide.

Anti-protozoal agents include atovaquone, benznidazole, clioquinol, decoquinate, diiodohydroxyquinoline, diloxamide furoate, dinitolmide, furazolidone, metronidazole, nimorazole, nitrofirazone, omidazole and tinidazole.

Anti-thyroid agents include drugs such as carbimazole, paricalcitol, and propylthiouracil.

Anti-tussives, include agents such as benzonatate.

Anxiolytics, sedatives, and hypnotics, include drugs such as alprazolam, amylobarbitone, barbitone, bentazepam, bromazepam, bromperidol, brotizolam, butobarbitone, carbromal, chlordiazepoxide, chlormethiazole, chlorpromazine, chlorprothixene, clonazepam, clobazam, clotiazepam, clozapine, dexmethylphenidate (d-threo-methylphenidate) diazepam, droperidol, ethinamate, flunanisone, flunitrazepam, triflupromazine, flupenthixol decanoate, fluphenazine, flurazepam, gabapentin, gaboxadol, .gamma.-hydroxybutyrate, haloperidol, lamotrigine, lorazepam, lormetazepam, medazepam, meprobamate, mesoridazine, methaqualone, methylphenidate, midazolam, modafinil, molindone, nitrazepam, olanzapine, oxazepam, pentobarbitone, perphenazine pimozide, pregabalin, prochlorperazine, pseudoephedrine, quetiapine, rispiridone, sertindole, siramesine, sulpiride, sunepitron, temazepam, thioridazine, triazolam, zaleplon, zolpidem, and zopiclone; appetite suppressants, anti-obesity drugs and drugs for treatment of eating disorders, such as amphetamine, bromocriptine, dextroamphetamine, diethylpropion, lintitript, mazindol, methamphetamine, orlistat, phentermine, and topiramate.

Cardiovascular drugs include angiotensin converting enzyme (ACE) inhibitors such as enalapril, ramipril, perindopril erbumine, 1-carboxymethyl-3-1-carboxy-3-phenyl-(1S)-propylamino-2,3,4,5-tetrahydro-1H-(3S)-1-benzazepine-2-one, 3-(5-amino-1-carboxy-1S-pentyl)amino-2,3,4,5-tetrahydro-2-oxo-3S-1H-1-benzazepine-1acetic acid or 3-(1-ethoxycarbonyl-3-phenyl-(1S)-propylamino)-2,3,4,5-tetrahydro-2-oxo-(−3 S)-benzazepine-1-acetic acid monohydrochloride; cardiac glycosides and cardiac inotropes such as amrinone, digoxin, digitoxin, enoximone, lanatoside C, medigoxin, and milrinone; calcium channel blockers such as verapamil, nifedipine, nicardipene, felodipine, isradipine, nimodipine, amlodipine and diltiazem; beta-blockers such as acebutolol, alprenolol, atenolol, labetalol, metoprolol, nadolol, oxyprenolol, pindolol, propafenone, propranolol, esmolol, sotalol, timolol, and acebutolol; antiarrhythmics such as moricizine, dofetilide, ibutilide, nesiritide, procainamide, quinidine, disopyramide, lidocaine, phenyloin, tocainide, mexiletine, flecainide, encainide, bretylium and amiodarone; cardioprotective agents such as dexrazoxane and leucovorin; vasodilators such as nitroglycerin; diuretic agents such as azetazolamide, amiloride, bendroflumethiazide, bumetamide, chlorothiazide, chlorthalidone, ethacrynic acid, furosemide, hydrochlorothiazide, metolazone, nesiritide, spironolactone, and triamterine; and miscellaneous cardiovascular drugs such as monteplase and corlopam; corticosteroids, such as beclomethasone, betamethasone, budesonide, cortisone, desoxymethasone, dexamethasone, fludrocortisone, flunisolide, fluocortolone, fluticasone propionate, hydrocortisone, methylprednisolone, prednisolone, prednisone and triamcinolone;

Erectile dysfunction drugs include apomorphine, phentolamine, and vardenafil.

Gastrointestinal agents, include drugs such as alosetron, bisacodyl, cilansetron, cimetidine, cisapride, diphenoxylate, domperidone, esomeprazole, famotidine, granisetron, lansoprazole, loperamide, mesalazine, nizatidine, omeprazole, ondansetron, prantoprazole, rabeprazole sodium, ranitidine, risperidone, sulphasalazine, and tegaserod; keratolytics, such as such as acetretin, calcipotriene, calcifediol, calcitriol, cholecalciferol, ergocalciferol, etretinate, retinoids, targretin, and tazarotene;

Lipid regulating agents include atorvastatin, bezafibrate, cerivastatin, ciprofibrate, clofibrate, ezetimibe, fenofibrate, fluvastatin, gemfibrozil, pitavastatin, pravastatin, probucol, rosuvastatin, and simvastatin;

Muscle relaxants, such as cyclobenzaprine, dantrolene sodium and tizanidine HCl;

Agents to treat neurodegenerative diseases, including active agents for treating Alzheimer's disease such as akatinol, donezepil, donepezil hydrochloride, dronabinol, galantamine, neotrofin, rasagiline, physostigmine, physostigmine salicylate, propentoffyline, quetiapine, rivastigmine, tacrine, tacrine hydrochloride, thalidomide, and xaliproden; active agents for treating Huntington's Disease, such as fluoxetine and carbamazepine; anti-parkinsonism drugs useful herein include amantadine, apomorphine, bromocriptine, entacapone, levodopa (particularly a levodopa/carbidopa combination), lysuride, pergolide, pramipexole, rasagiline, riluzole, ropinirole, selegiline, sumanirole, tolcapone, trihexyphenidyl, and trihexyphenidyl hydrochloride; and active agents for treating ALS such as the anti-spastic agents baclofen, diazemine, and tizanidine.

Nitrates and other anti-anginal agents, include drugs such as amyl nitrate, glyceryl trinitrate, isosorbide dinitrate, isosorbide mononitrate and pentaerythritol tetranitrate.

Neuroleptic drugs, including antidepressant drugs, antimanic drugs, and antipsychotic agents, wherein antidepressant drugs include (a) the tricyclic antidepressants such as amoxapine, amitriptyline, clomipramine, desipramine, doxepin, imipramine, maprotiline, nortriptyline, protriptyline, and trimipramine, (b) the serotonin reuptake inhibitors citalopram, fluoxetine, fluvoxamine, paroxetine, sertraline, and venlafaxine, (c) monoamine oxidase inhibitors such as phenelzine, tranylcypromine, and (−)-selegiline, and (d) other antidepressants such as aprepitant, bupropion, duloxetine, gepirone, igmesine, lamotrigine, maprotiline, mianserin, mirtazapine, nefazodone, rabalzotan, sunepitron, trazodone and venlafaxine, and wherein antimanic and antipsychotic agents include (a) phenothiazines such as acetophenazine, acetophenazine maleate, chlorpromazine, chlorpromazine hydrochloride, fluphenazine, fluphenazine hydrochloride, fluphenazine enanthate, fluphenazine decanoate, mesoridazine, mesoridazine besylate, perphenazine, thioridazine, thioridazine hydrochloride, trifluoperazine, and trifluoperazine hydrochloride, (b) thioxanthenes such as chlorprothixene, thiothixene, and thiothixene hydrochloride, and (c) other heterocyclic drugs such as carbamazepine, clozapine, droperidol, haloperidol, haloperidol decanoate, loxapine succinate, molindone, molindone hydrochloride, olanzapine, pimozide, quetiapine, risperidone, and sertindole.

Nutritional agents include calcitriol, carotenes, dihydrotachysterol, essential fatty acids, non-essential fatty acids, phytonadiol, vitamin A, vitamin B.sub.2, vitamin D, vitamin E and vitamin K.

Opioid analgesics include alfentanil, apomorphine, buprenorphine, butorphanol, codeine, dextropropoxyphene, diamorphine, dihydrocodeine, fentanyl, hydrocodone, hydromorphone, levorphanol, meperidine, meptazinol, methadone, morphine, nalbuphine, oxycodone, oxymorphone, pentazocine, propoxyphene, sufentanil, and tramadol.

Stimulants include active agents for treating narcolepsy, attention deficit disorder (ADD) and attention deficit hyperactivity disorder (ADHD), such as amphetamine, dexamphetamine, dexfenfluramine, fenfluramine, mazindol, methylphenidate (including d-threo-methylphenidate, or “dexmethylphenidate,” as well as racemic d,l-threo-methylphenidate), modafinil, pemoline, and sibutramine.

Pharmaceutically acceptable, pharmacologically active salts and derivatives of the active ingredients, including enantiomers and their pharmaceutically acceptable salts, mixtures of enantiomers and their pharmaceutically acceptable salts, and active metabolites and their pharmaceutically acceptable salts may be used in the compositions.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, and isethionic.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “stereoisomers” refers to compounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. As used herein, the term “optical isomer” is equivalent to the term “enantiomer”. The terms “racemate”, “racemic mixture” or “racemic modification” refer to a mixture of equal parts of enantiomers. The term “chiral center” refers to a carbon atom to which four different groups are attached. The term “enantiomeric enrichment” as used herein refers to the increase in the amount of one enantiomer as compared to the other. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art using standard techniques well known in the art, such as those described by J. Jacques, et al., “Enantiomers. Racemates, and Resolutions”, John Wiley and Sons, Inc., 1981. Examples of resolutions include recrystallization of diastereomeric salts/derivatives or preparative chiral chromatography.

C. Excipients

Formulations may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein, “carrier” includes, but is not limited to, plasticizers, anti-tacking agents, diluents, binders, lubricants, disintegrants, stabilizers, surfactants, colorants, and fillers.

The plasticizer could be either a solid plasticizer or liquid plasticizer. Suitable solid plasticizers include, but are not limited to, polyethylene glycols having molecular weight between 1500 to 8000, block co-polymers of ethylene oxide and propylene oxide (EO/PO) available under the tradename Pluronic®, and mixtures thereof. In one embodiment, the solid plasticizer is polyethylene glycol 3350 (PEG 3350) or polyethylene glycol 4000 (PEG 4000).

A solid plasticizer can be included in the polymer blend composition at a concentration from about 1% to about 20% by weight of the polymer blend coating composition, more preferably from about 1% to about 18% by weight of the dry coating composition.

Representative liquid plasticizers include triethylcitrate, glyceryl triacetate, acetyltriethylcitrate, dibutyl sebacate, diethyl phthalate, polyethylene glycol 400, glycerol, castor oil, or mixtures thereof.

A liquid plasticizer can be included in the polymer blend composition in a range of greater than 0% to about 20% by weight of the polymer blend composition.

The anti-sticking agents used in the film coating of the pharmaceutical composition may be chosen from those known in the pharmaceutical art. In a preferred embodiment, the anti-sticking agents are talc, stearic acid, sodium stearate, magnesium stearate, sodium stearyl fumarate, silicon dioxide, glycerol monostearate, or glyceryl behenate.

Further ingredients for film coating are dyes such as, for example, iron oxides or quinoline yellow, wetting agents such as, for example, sodium lauryl sulfate or Cremophor RH 40, and antifoams such as, for example, simethicone.

Solvents may be chosen from those known in the pharmaceutical art. In a preferred embodiment, the solvents are water, alcohol, acetone, isopropanol, dichloromethane or combinations thereof.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose e and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, anti-oxidants, or preservatives.

II. Method of Making Taste-Masked Compositions

The taste masked compositions can be in the form of tablets, capsules, particles, beads, powders, and troches, or in a liquid suspension. The taste masking polymer combination can be applied to the exterior of these devices, or to the particles, powder, beads or granules which are compressed or bound to form the devices.

For example, particles of the active ingredient may be spray coated with the polymer coating either directly or after granulation. The coated particles can then be admixed with other pharmaceutically acceptable additives such as sweeteners or flavorings in an aqueous liquid vehicle for oral administration.

The preparation of the formulation may be accomplished by a variety of coating techniques known in the art including fluidized bed coating, conventional top spray coating and wet granulation techniques. Preferably, fluidized bed coating with a Wurster column insert is used to apply the coating. In this procedure, the particles of active agent or active containing beads to be coated are suspended in an apparatus that creates an upward stream of air in which the particles move. The stream passes through an area of finely atomized coating material which causes the passing particles to be coated, after which the coated particles move upward through the Wurster column and then travel downward in a fluidized condition countercurrent to a flow of heated fluidized gas whereupon they are dried. The particles may reenter the upward stream for further coating.

Generally, the polymer coating material is dissolved in either an organic solvent or water to make a solution or suspension for use in the fluidized bed coating process. A variety of organic solvents may be used. The solvent is removed in the drying process and is thus not present in the final composition. The total polymer concentration in the coating solutions can vary, generally in the range of about 3 to about 20% by weight, preferably in range about 5-15% w/w.

Once the dried coated particles or beads are obtained, the coated particles or beads are admixed with other pharmaceutically acceptable adjuvants such as flavorings, sweeteners, thickening agents, colorings and the like to form compositions for oral liquid administration. Suitable flavorants include fruit flavors, peppermint, licorice or bubble gum flavors. The sweetening agents may be for example bulk sweeteners such as sucrose or polyols (e.g. maltitol, sorbitol) and/or intense sweeteners such as saccharin, aspartame or acesulfame K. The preparation can be formed as a liquid, or as a powder for reconstitution with water by the pharmacist prior to dispensing.

The taste masking formulations satisfy the unique requirements of a liquid formulation. The formulation is stable in an aqueous environment after reconstitution while still providing appropriate taste masking when the product is administered.

III. Methods of Use

The formulation can be administered to any patient in need thereof. The amount of the active ingredient(s) to be administered is determined based on the amount which provides the desired dose to the patient in need of such treatment to alleviate symptoms or treat a condition. The compositions will typically be administered orally. The composition can be administered in a single dose, an escalating dose, or administered at an elevated dosage which is then decreased to a lower dosage after a particular circulating blood concentration of the ingredient(s) has been achieved. One of skill in the art would be able to choose administration protocols and determine appropriate dosing regimes to treat the various disorders. For many of the disclosed compounds, appropriate dosage ranges have been established to maximize circulating concentrations of the compound and minimize side-effects.

### EXAMPLE

The present invention will be further understood by reference to the following non-limiting example.

### Example 1

### Preparation of Taste Masked Doxycycline Composition for Oral Liquid Administration

Mass

Composition of Formulation A (027-17EC)

| | | |
|---|---|---|
| Core Composition | | |
| Non pareil seed cores: | 1200 | mg |
| Doxycycline hyclate | 1056 | mg |
| Povidone USP | 144 | mg |
| Hydroxypropyl methylcellulose | 72 | mg |
| Hybrid Core Coating Composition | | |
| Hydroxypropyl Methylcellulose phthalate | 117 | mg |
| Ammonio Methylacrylate copolymer Type A | 117 | mg |
| Triethyl Citrate | 23 | mg |
| Talc | 84 | mg |

Composition of Formulation B (025-56DC)

| | | |
|---|---|---|
| Core Composition | | |
| Non pareil seed cores: | 1200 | mg |
| Doxycycline hyclate | 1056 | mg |
| Povidone USP | 144 | mg |
| Hydroxypropyl methylcellulose | 72 | mg |
| Hybrid Core Coating Composition | | |
| Ammonio Methylacrylate copolymer Type A | 216 | mg |
| Methylacrylic acid copolymer Type C | 324 | mg |
| Triethyl Citrate | 73 | mg |

Composition of Formulation C (025-64CC)

| | | |
|---|---|---|
| Core Composition | | |
| Non pareil seed cores: | 1200 | mg |
| Doxycycline hyclate | 1056 | mg |
| Povidone USP | 144 | mg |
| Hydroxypropyl methylcellulose | 72 | mg |
| Hybrid Core Coating Composition | | |
| Ammonio Methylacrylate copolymer Type A | 108 | mg |
| Ammonio Methylacrylate copolymer Type B | 108 | mg |
| Methylacrylic acid Copolymer Type C | 216 | mg |
| Triethyl Citrate | 43 | mg |
| Talc | 173 | mg |

Non-pareil sugar seeds were placed in a fluidizer bed and coated with a solution or dispersion of active ingredient with binder components to form drug layered pellets. The drug layered pellets were then coated with an aqueous dispersion containing a polymer combination of an enteric polymer and an methacrylate copolymer, as described for each of the above-identified formulations.

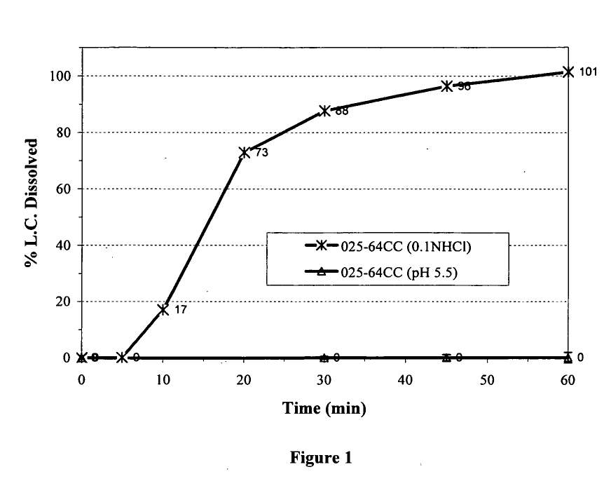

The coated doxycycline beads were tested using a dissolution apparatus. Dissolution studies were conducted in aqueous medium at pHs 1.2 and 5.5. A sample of known volume was withdrawn at designated time intervals from the vessel and subjected to a suitable assay procedure. The percentage of doxycycline released as a function of time is plotted as the dissolution profile. The dissolution profiles for Formulations A, B, and C at pHs 1.2 and 5.5 are shown in FIGS. 1-4.

FIG. 1 shows the dissolution profile of Formulation C (025-64CC) at a pH of 1.2 and 5.5. The graph show no release of doxycycline at a pH of 5.5. However, at a pH of 1.2, the drug is released rapidly over one hour.

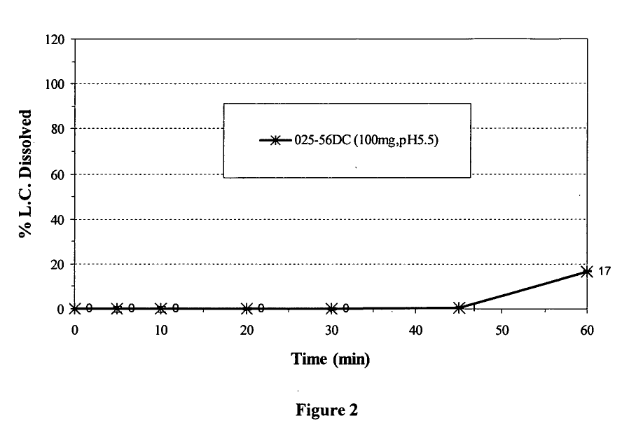

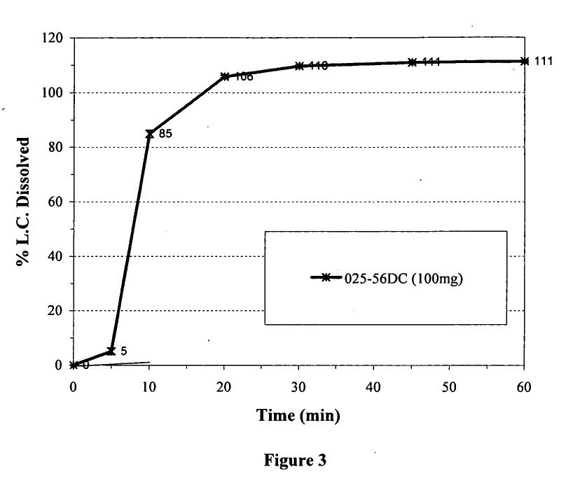

FIGS. 2 and 3 show the dissolution profile for Formulation B (025-56DC) at a pH of 5.5 and 1.2, respectively. At a pH of 5.5 (FIG. 2), little or no doxycycline is released over one hour. At a pH of 1.2 (FIG. 3), however, doxycycline is released rapidly over one hour.

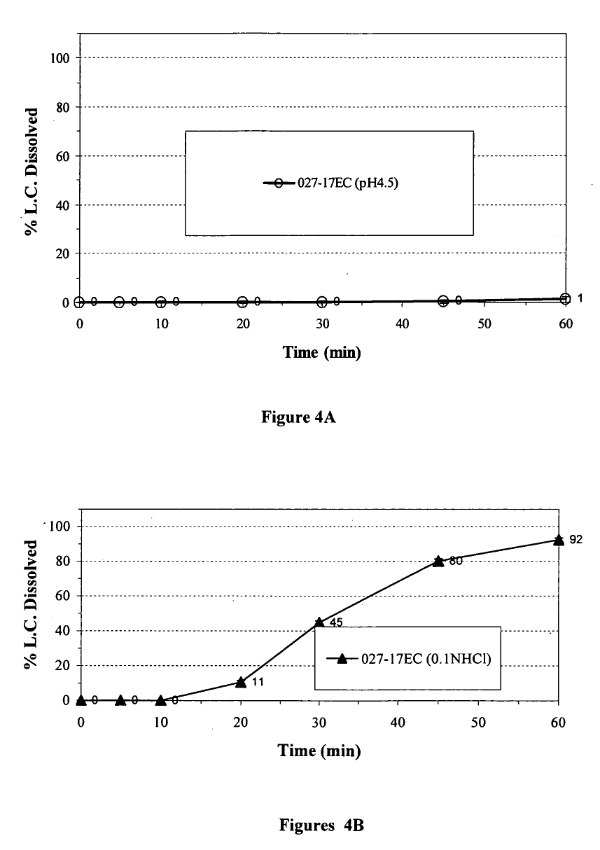

FIG. 4 shows the the dissolution profile for Formulation A (027-17EC) at a pH of 1.2 (FIG. 4b) and 4.5 (FIG. 4a). At a pH of 4.5, little or no doxycycline is released over one hour. At a pH of 1.2, however, doxycycline is released rapidly over one hour.

The results, as shown in FIGS. 1-4, demonstrate that very little active agent is released at pH 5.5, while rapid release was observed at 0.1N HCl medium.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of skill in the art to which the disclosed invention belongs.

We claim:

1. A pharmaceutical composition for oral administration comprising a pharmaceutically active ingredient coated with a taste masking polymer combination comprising an enteric polymer and an ammonio methacrylate copolymer, in an amount effective to mask the taste of the medicament.

2. The pharmaceutical composition of claim 1 wherein the taste masking polymer combination is a blend.

3. The pharmaceutical composition of claim 1 wherein the taste masking polymer combination is substantially insoluble at the pH in the mouth, but dissolves at the pH of the stomach or intestine.

4. The pharmaceutical composition of claim 1, wherein the polymer weight ratio of the enteric polymer to the ammonio methacrylate copolymer is between about 40:60 to about 90:10.

5. The pharmaceutical composition of claim 4 wherein the polymer weight ratio of the enteric polymer to the ammonio methacrylate copolymer is about 60:40.

6. The pharmaceutical composition of claim 1 wherein the ammonio methacylate copolymer is a copolymer of acrylate and methacrylate with a quaternary ammonium group.

7. The pharmaceutical composition of claim 1 wherein the enteric polymer dissolves at a pH of 5.0 or higher and is selected from the group consisting of microcrystalline cellulose, cellulose acetate, cellulose succinate, cellulose phthalate, hydroxypropylmethyl cellulose acetate, hydroxypropylmethyl succinate, hydroxypropylmethyl phthalate, cellulose acetate trimellitate, cellulose acetate phthalate, acrylic acid polymers and copolymers other than ammonio methacrylate copolymers, polyvinylacetate phthalate, copoly(ethylene vinylacetate) (EVAC), maleic anhydride-co-alkylene copolymers, polyalkylene oxides, and mixtures thereof.

8. The pharmaceutical composition of claim 7 wherein the acrylic acid polymer or copolymer is selected from the group consisting of methacrylic acid copolymer Type A, methacrylic acid copolymer Type B, methacrylic acid copolymer Type C, and combinations thereof.

9. The pharmaceutical composition of claim 1 wherein the composition is a liquid formulation containing particles of active medicament coated with the taste masking polymer combination.

10. The pharmaceutical composition of claim 1 wherein the taste masking polymer combination comprises about 3 to 120% by weight of the solid substrate.

11. The pharmaceutical composition of claim 10 wherein the taste masking polymer combination comprises about 5-40% by weight of the solid substrate.

12. The pharmaceutical composition of claim 1 wherein the ammonio methacrylate copolymer is selected from highly permeable copolymers of acrylates and methacrylates with quaternary ammonium groups and poorly permeable copolymers of acrylates and methacrylates with quaternary ammonium groups.

13. The pharmaceutical composition of claim 1 comprising a solid plasticizer selected from the group consisting of polyethylene glycol having a molecular weight of 1500 to 8000, block co-polymers of ethylene oxide and propylene oxide (EO/PO), and mixtures thereof.

14. The pharmaceutical composition of claim 13 wherein the concentration of the plasticizer is from about 1% to about 20% by weight of the polymer blend coating composition.

15. The pharmaceutical composition of claim 14 wherein the concentration of the plasticizer is from about 1% to about 18% by weight of the dry coating composition.

16. The pharmaceutical composition of claim 1 comprising a liquid plasticizer selected from the group consisting of triethylcitrate, glyceryl triacetate, acetyltriethylcitrate, dibutyl sebacate, diethyl phthalate, polyethylene glycol 400, glycerol, castor oil, and mixtures thereof.

17. The pharmaceutical composition of claim 16 wherein the concentration of the liquid plasticizer is from greater than 0% to about 20% by weight of the polymer blend composition.

18. The pharmaceutical composition of claim 1 wherein the formulation is in a form selected from the group consisting of tablets, capsules, beads, pellets, powder, granules, crystals, particles, and troches.

19. The pharmaceutical composition of claim 1 wherein the active ingredient is selected from the group consisting of antibiotic drugs, analgesic drugs, anti-inflammatory drugs, gastro-intestinal drugs, antihistamines, decongestants, anti-depressants, anti-psychotics, antivirals, oncolytics, vaccines, antiepileptics, ant-asthma drugs, and antispasmodics. β-adrenergic receptor blockers, alkaloids, antacids, analgesics, anabolic agents, anti-anginal drugs, anti-allergy agents, anti-angiogenesis agents, anti-arrhythmia agents, antiasthmatics, antibiotics, anticholesterolemics, anticonvulsants, anticoagulants, antidepressants, antidiarrheal preparations, anti-emetics, anti-epileptics, antihistamines, antihypertensives, anti-infectives, anti-inflammatory agens, antihyperlipidemic drugs, antimanics, anti-migraine agents, antinauseants, anti-parkinsonism drugs, antipsychotics, antistroke agents, antithyroid preparations, anabolic drugs, antiobesity agents, antiparasitics, antipsychotics, antipyretics, antispasmodics, antithrombotics, antitumor agents, antitussives, antiulcer agents, anti-uricemic agents, antiviral drugs, anxiolytic agents, appetite stimulants, appetite suppressants, beta-blocking agents, bronchodilators, calcium antagonists, cardiotonic drugs, cardiovascular agents, cerebral dilators, chelating agents, cholecystekinin antagonists, chemotherapeutic agents, cholesterol reducing agents, cognition activators, contraceptives, coronary vasodilators, cough suppressants, CNS drugs, decongestants, diabetes agents, diuretics, drugs for erectile dysfunction, emollients, enzymes, erythropoietic drugs, expectorants, fertility agents, fungicides, gastrointestinal agents, gout treating drugs, growth regulators, hormone drugs, hyperglycemic agents, hypnotic drugs, hypoglycemic agents, ion-exchange resins, laxatives, migraine treatments, mineral supplements, mucolytics, narcotics, neuroleptics, neuromuscular drugs, non-steroidal anti-inflammatories (NSAIDs), nutritional additives, osteoporosis treating agents, peripheral vasodilators, polypeptides, prostaglandins, psychotropics, renin inhibitors, respiratory stimulants, sedatives, serotonin receptor antagonists, steroidal anti-inflammatory drugs, steroids, stimulants, sympatholytics, thyroid preparations, tranquilizers, uterine relaxants, vaccines, vaginal preparations, vasoconstrictors, vasodilators, vertigo agents, vitamins, wound healing agents, and combinations thereof.

20. A method of masking the taste of a medicament comprising applying a coating or film of a combination of an enteric polymer and an ammonio methacrylate copolymer to an active ingredient in an amount effective to mask the taste of the active ingredient.

21. The method of claim 20 wherein the taste masking polymer combination is a blend.

22. The method of claim 20 wherein the taste masking polymer combination is substantially insoluble at the pH in the mouth, but dissolves at the pH of the stomach or intestine.

23. The method of claim 20, wherein the polymer weight ratio of the enteric polymer to the ammonio methacrylate copolymer is between about 40:60 to about 90:10.

24. The method of claim 23 wherein the polymer weight ratio of the enteric polymer to the ammonio methacrylate copolymer is about 60:40.

25. The method of claim 20 wherein the ammonio methacylate copolymer is a copolymer of acrylate and methacrylate with a quaternary ammonium group.

26. The method of claim 20 wherein the enteric polymer dissolves at a pH of 5.0 or higher and is selected from the group consisting of microcrystalline cellulose, cellulose acetate, cellulose succinate, cellulose phthalate, hydroxypropylmethyl cellulose acetate, hydroxypropylmethyl succinate, hydroxypropylmethyl phthalate, cellulose acetate trimellitate, cellulose acetate phthalate, acrylic acid polymers and copolymers other than ammonio methacrylate copolymers, polyvinylacetate phthalate, copoly(ethylene vinylacetate) (EVAC), maleic anhydride-co-alkylene copolymers, polyalkylene oxides, and mixtures thereof.

27. The method of claim 26 wherein the acrylic acid polymer or copolymer is selected from methacrylic acid copolymer Type A, methacrylic acid copolymer Type B, and methacrylic acid copolymer Type C,

28. The method of claim 20 wherein the composition is a liquid formulation containing particles of active medicament coated with the taste masking polymer combination.

29. The method of claim 20 wherein the taste masking polymer combination comprises about 3 to 120% by weight of the solid substrate.

30. The method of claim 29 wherein the taste masking polymer combination comprises about 5-40% by weight of the solid substrate.

31. The method of claim 20 wherein the ammonio methacrylate copolymer is selected from highly permeable copolymers of acrylates and methacrylates with quaternary ammonium groups and poorly permeable copolymers of acrylates and methacrylates with quaternary ammonium groups.

32. The method of claim 20 comprising a solid plasticizer selected from the group consisting of polyethylene glycol having a molecular weight of 1500 to 8000, block co-polymers of ethylene oxide and propylene oxide (EO/PO), and mixtures thereof.

33. The method of claim 32 wherein the concentration of the plasticizer is from about 1% to about 20% by weight of the polymer blend coating composition.

34. The method of claim 33 wherein the concentration of the plasticizer is from about 1% to about 18% by weight of the dry coating composition.

35. The method of claim 20 comprising a liquid plasticizer selected from the group consisting of triethylcitrate, glyceryl tri acetate, acetyltriethylcitrate, dibutyl sebacate, diethyl phthalate, polyethylene glycol 400, glycerol, castor oil, and mixtures thereof.

36. The pharmaceutical composition of claim 35 wherein the concentration of the liquid plasticizer is from greater than 0% to about 20% by weight of the polymer blend composition.

37. The method of claim 20 wherein the formulation is in a form selected from the group consisting of tablets, capsules, beads, pellets, powder, granules, crystals, particles, and troches.

38. The method of claim 20 wherein the active ingredient is selected from the group consisting of antibiotic drugs, analgesic drugs, anti-inflammatory drugs, gastro-intestinal drugs, antihistamines, decongestants, anti-depressants, anti-psychotics, antivirals, oncolytics, vaccines, antiepileptics, ant-asthma drugs, and antispasmodics. β-adrenergic receptor blockers, alkaloids, antacids, analgesics, anabolic agents, anti-anginal drugs, anti-allergy agents, anti-angiogenesis agents, anti-arrhythmia agents, antiasthmatics, antibiotics, anticholesterolemics, anticonvulsants, anticoagulants, antidepressants, antidiarrheal preparations, anti-emetics, anti-epileptics, antihistamines, antihypertensives, anti-infectives, anti-inflammatory agens, antihyperlipidemic drugs, antimanics, anti-migraine agents, antinauseants, anti-parkinsonism drugs, antipsychotics, antistroke agents, antithyroid preparations, anabolic drugs, antiobesity agents, antiparasitics, antipsychotics, antipyretics, antispasmodics, antithrombotics, antitumor agents, antitussives, antiulcer agents, anti-uricemic agents, antiviral drugs, anxiolytic agents, appetite stimulants, appetite suppressants, beta-blocking agents, bronchodilators, calcium antagonists, cardiotonic drugs, cardiovascular agents, cerebral dilators, chelating agents, cholecystekinin antagonists, chemotherapeutic agents, cholesterol reducing agents, cognition activators, contraceptives, coronary vasodilators, cough suppressants, CNS drugs, decongestants, diabetes agents, diuretics, drugs for erectile dysfunction, emollients, enzymes, erythropoietic drugs, expectorants, fertility agents, fungicides, gastrointestinal agents, gout treating drugs, growth regulators, hormone drugs, hyperglycemic agents, hypnotic drugs, hypoglycemic agents, ion-exchange resins, laxatives, migraine treatments, mineral supplements, mucolytics, narcotics, neuroleptics, neuromuscular drugs, non-steroidal anti-inflammatories (NSAIDs), nutritional additives, osteoporosis treating agents, peripheral vasodilators, polypeptides, prostaglandins, psychotropics, renin inhibitors, respiratory stimulants, sedatives, serotonin receptor antagonists, steroidal anti-inflammatory drugs, steroids, stimulants, sympatholytics, thyroid preparations, tranquilizers, uterine relaxants, vaccines, vaginal preparations, vasoconstrictors, vasodilators, vertigo agents, vitamins, wound healing agents, and combinations thereof.
